(19) United States
(12) Patent Application Publication
(10) Pub. No.: US 2006/0270981 A1
(43) Pub. Date: Nov. 30, 2006

(54) Coiled injection tube

(76) Inventors: Leonilda Capuano, Pointe-Claire (CA); Robert D. Martin, Lutz, FL (US); Teresa Ann Mihalik, Montreal (CA)

Correspondence Address: John Christopher;Christopher & Weisberg, P.A., Suite 2040, 200 East Las Olas Boulevard, Fort Lauderdale, FL, 33301, US

(21) Appl. No.: 11/129,044

(22) Filed: May 13, 2005

(51) Int. Cl. A61F 7/12; A61M 29/00

(52) U.S. Cl. 604/113; 604/96.01

(57) ABSTRACT

A medical device includes a catheter having a proximal end and a distal end, the catheter defining a lumen. A passage for a guide wire is disposed within the lumen so as to be coaxial with the lumen. The passage has an open proximal end that is substantially coterminous with the proximal end of the catheter and an open distal end that is substantially coterminous with the distal end of the catheter. A fluid injection tube is wound around the passage for the guide wire and is provided with radially spaced fluid injection ports.

### CROSS-REFERENCE TO RELATED APPLICATION

n/a

### STATEMENT REGARDING FEDERALLY SPONSORED RESEARCH OR DEVELOPMENT

n/a

### FIELD OF THE INVENTION

The present invention relates to a medical device, and more particularly to a coolant injection tube for a thermal treatment medical device.

### BACKGROUND OF THE INVENTION

Medical devices are known for thermally treating tissue on the exterior and the interior of the body. One category of such devices is the minimally-invasive, catheter-based device that is introduced into the vasculature. One feature of these devices is means by which the device is positioned at the treatment site. For example, some devices are actively steered through the vasculature using a steering or deflection mechanism, such as a pull-wire; whereas other devices are introduced over a wire that has already been guided to a selected location, wherein the wire acts as a guide that leads the device to the treatment site. Although a device can be configured so that the guiding wire is substantially external to the device, most known devices include a central longitudinal lumen that receives the wire.

Another feature of the minimally-invasive, catheter-based, thermal-treatment device is the thermal treatment mechanism. One category of devices thermally treats tissue by cooling it, wherein cooling is effected by injecting coolant into a portion of the device, such as a distal device portion that has advantageous thermal transfer properties, and placing the distal device portion near or in contact with the tissue. The distal end portion can have a fixed diameter that is substantially the same as the diameter of the remainder of the catheter or it can have a variable diameter, such as is provided by a balloon. However, regardless of whether the whether the distal end portion is of fixed or variable diameter, the overall size of the device and the injection tube are limited by the dimensions of the vasculature. Typical devices are 5 mm to 7 mm in diameter. Given the small device size, it has proven challenging to cool or freeze warm bodily tissue to a temperature near or well below freezing. Accordingly, it is important to maximize the cooling potential of the coolant by delivering or injecting it at a particular location within the device.

In order to cool other than a small spot, devices as depicted in U.S. Pat. No. 6,235,019 provide multiple coolant injection tubes. Alternately, as shown in U.S. Pat. No. 5,899,898, a single injection tube can be provided with openings along its length. Although such coolant injection structures can be very desirable for created an elongated cooling zone, they are less suitable for balloon devices or over-the-wire devices. With respect to an over-the-wire device, it will be noted that a relatively large central passage for the wire actually blocks or isolates the injection lying at one side of the passage from the opposite side of the passage, thereby insulating the masked side of the device and creating uneven cooling.

Although not directed to issues related to an over-the-wire device, U.S. Pat. No. 6,551,274 illustrates a loosely coiled injection tube. However, as with the linear injection tubes having a series of longitudinal ports, at regular intervals along the device, the central structure masks the injection tube.

In view of the preceding, it is believed that an improved injection tube would be desirable for use with over-the-wire devices or other devices that have structures other than an injection tube in the cooling chamber of the device.

### SUMMARY OF THE INVENTION

The present invention provide an improved injection tube for use with over-the-wire devices or devices that have structures other than an injection tube in the cooling chamber of the device. In an exemplary embodiment a medical includes a steering element and a fluid injection tube, wherein a portion of the fluid injection tube is wound around the steering element. The steering element can include a passage for a guide wire.

More particularly, the medical device includes a catheter having a proximal end and a distal end, the catheter defining a lumen; a passage for a guide wire disposed within the lumen so as to be coaxial with the lumen and having an open proximal end that is substantially coterminous with the proximal end of the catheter and an open distal end that is substantially coterminous with the distal end of the catheter; a fluid injection tube, wherein a portion of the fluid injection tube is wound around the passage for the guide wire; and a plurality of radially spaced injection ports in the portion of the fluid injection tube that is wound around the passage for the guide wire.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is side section view showing the interior of a catheter based medical device in accordance with the invention; and

FIG. 2 illustrates an alternate embodiment of the catheter based medical device shown in FIG. 1.

### DETAILED DESCRIPTION OF THE INVENTION

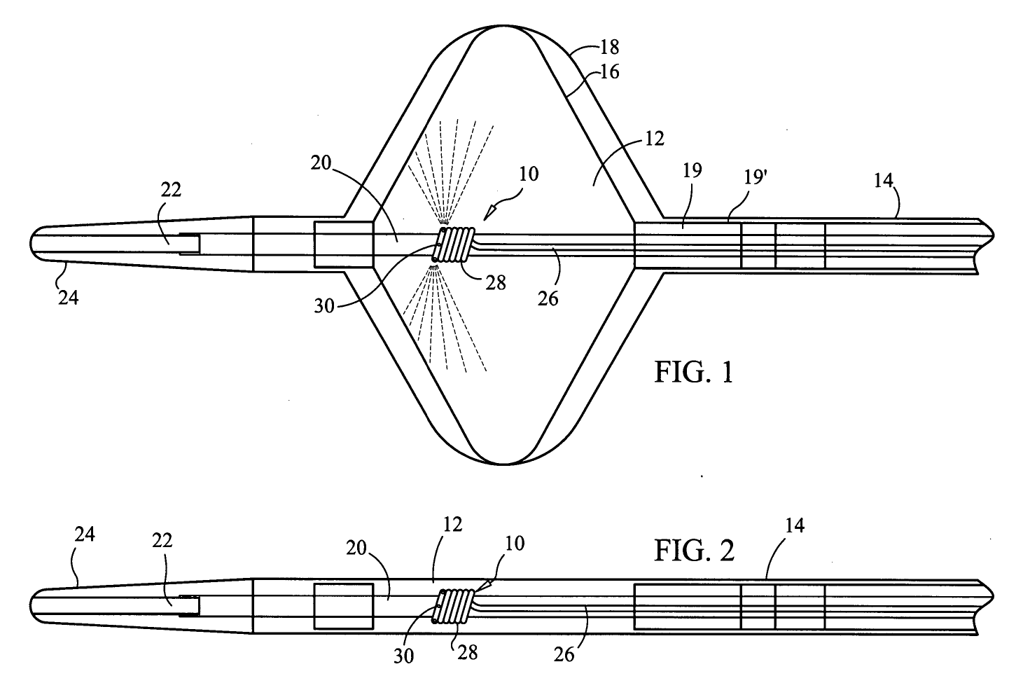

Referring to FIGS. 1 and 2, a medical device in accordance with the invention is illustrated. The device includes a fluid injection tube 10 disposed within a lumen 12, space or void defined by a portion of a catheter 14. As shown in FIG. 1, the lumen 12 is defined by a first balloon 16 encapsulated within a second balloon 18. The second balloon 18 contains leaks in the first balloon 16 should they occur and the gap shown between the balloons for the purposes of illustration do not exist when the inner balloon is inflated. Low pressure or vacuum return lumens 19 and 19′ are in fluid communication with the interior of the first and second balloons, respectively.

However, referring to FIG. 2, the lumen 12 can also be a substantially uniform diameter passage within a wall portion of the catheter 14, one lumen of a multi-lumen configuration, or central lumen within a catheter that is coaxial with the longitudinal axis of the catheter.

At least a portion of the fluid injection tube 10 is wound around a structure 20 that passes through or is contained within the lumen 12 such as another tube, a wire, a shim, or a spring. In the illustration of FIGS. 1 and 2, the structure 20 is part of a catheter steering element, namely, a tube that defines a passage 22 or lumen for a guide wire (not shown). As shown, the passage 22 has an open proximal end that is substantially coterminous with the proximal end of the catheter and an open distal end that is substantially coterminous with the distal end of the catheter. The guide wire is suitable for placement into the vasculature of a patient and the passage 22 slides over the wire (i.e., the wire goes through the passage), for guiding the distal portion of the catheter to a desired location using techniques known in the art. The distal end of the catheter can include a soft tip element 24.

Continuing to refer to FIGS. 1 and 2, the fluid injection tube 10 includes a longitudinal portion 26 in fluid communication with a helically wound portion 28, wherein the helically wound portion 28 is wrapped around a portion of the passage 22 and the longitudinal portion 26 is disposed adjacent and exterior to the passage 22. In an exemplary embodiment, the helically wound portion 28 includes at two or more windings (in the illustrated embodiment there are six windings) that span an axial distance along the passage 22 about 0.1 inches. Although the fluid injection tube 10 can be tacked or firmly bonded to the exterior of the passage 22, it can alternatively be secured to the passage 22 only by the encirclement thereof by the helically wound portion 28 so that the fluid injection tube and the passage can be axially movable with respect to each other.

The fluid injection tube 10 can be apertured or plugged at its distal end, and/or it can include multiple radially-spaced injection ports 30 helically wound portion 28. The radially spaced injection ports 30 can be equally spaced apart; and, in an exemplary embodiment, four injection ports 30 are spaced 90 degrees apart on the distalmost winding. As to materials, the longitudinal portion 26 can be made of polyimide and helically wound portion 28 can be made of stainless steel. When coolant in liquid, gas, or mixed phase state exits the ports 30 (as shown by a stylized spray pattern), the coolant expands and/or fills the lumen or space 12 and then is evacuated through the return lumen 19.

In an exemplary embodiment, the longitudinal portion 26 includes 0.0126″ polyimide tubing and helically wound portion 28 is a stainless steel coil having a 0.022″ outer diameter and a 0.014 inner diameter. There are four 0.0025″ laser drilled ports in the helically wound portion that are equally spaced.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A medical device for thermally affecting tissue comprising:
a steering element; and
a fluid injection tube, wherein a portion of the fluid injection tube is wound around the steering element.

2. The medical device of claim 1, wherein the steering element includes a passage for a guide wire.

3. The medical device of claim 2, further comprising a catheter having a proximal end and a distal end, the catheter defining a lumen, wherein the passage for the guide wire is disposed within the lumen of the catheter.

4. The medical device of claim 3, wherein the passage for the guide wire is coaxial with the longitudinal axis of the lumen.

5. The medical device of claim 4, wherein the passage for the guide wire has an open proximal end that is substantially coterminous with the proximal end of the catheter and an open distal end that is substantially coterminous with the distal end of the catheter.

6. The medical device of claim 3, wherein the fluid injection tube has a plurality of radially spaced injection ports in the portion of the fluid injection tube that is wound around the passage for the guide wire.

7. The medical device of claim 6, wherein the fluid injection tube includes a longitudinal portion in fluid communication with the portion of the fluid injection tube that is wound around the passage for the guide wire, wherein the longitudinal portion is disposed adjacent and exterior to the passage for the guide wire.

8. The medical device of claim 6, wherein the plurality of radially spaced injection ports are equally spaced apart.

9. The medical device of claim 8, wherein there are four injection ports.

10. The medical device of claim 6, wherein the longitudinal portion of the fluid injection tube is made of polyimide and the portion of the fluid injection tube that is wound around the passage for the guide wire is made of stainless steel.

11. The medical device of claim 6, wherein the portion of the fluid injection tube that is wound around the passage for the guide wire includes at least two windings and wherein the radially spaced injection ports are in the distalmost winding.

12. The medical device of claim 11, wherein the at least two windings span an axial distance along the passage for the guide wire of about 0.1 inches.

13. A medical device for thermally affecting tissue comprising:
a passage for a guide wire;
a fluid injection tube, wherein a portion of the fluid injection tube is wound around the passage for the guide wire; and
a plurality of radially spaced injection ports in the portion of the fluid injection tube that is wound around the passage for the guide wire.

14. The medical device of claim 13, wherein the fluid injection tube includes a longitudinal portion in fluid communication with the portion of the fluid injection tube that is wound around the passage for the guide wire, wherein the longitudinal portion is disposed adjacent and exterior to the passage for the guide wire.

15. The medical device of claim 14, wherein the plurality of radially spaced injection ports are equally spaced apart.

16. The medical device of claim 13, wherein there are four injection ports.

17. The medical device of claim 13, wherein the longitudinal portion of the fluid injection tube is made of polyimide and the portion of the fluid injection tube that is wound around the passage for the guide wire is made of stainless steel.

18. The medical device of claim 17, wherein the portion of the fluid injection tube that is wound around the passage for the guide wire includes at least two windings and wherein the radially spaced injection ports are in the distalmost winding.

19. The medical device of claim 18, wherein the at least two windings span an axial distance along the passage for the guide wire of about 0.1 inches.

20. A medical device for thermally affecting tissue comprising:
a catheter having a proximal end and a distal end, the catheter defining a lumen;
a passage for a guide wire disposed within the lumen so as to be coaxial with the lumen and having an open proximal end that is substantially coterminous with the proximal end of the catheter and an open distal end that is substantially coterminous with the distal end of the catheter;
a fluid injection tube, wherein a portion of the fluid injection tube is wound around the passage for the guide wire; and
a plurality of radially spaced injection ports in the portion of the fluid injection tube that is wound around the passage for the guide wire.
